(19) United States
(12) Patent Application Publication
(10) Pub. No.: US 2018/0340164 A1
(43) Pub. Date: Nov. 29, 2018

(54) METHOD FOR INCREASING GROWTH AND METABOLISM EFFICIENCY OF RECOMBINANT MICROORGANISM UNDER ANAEROBIC ENVIRONMENT

(71) Applicant: National Chung Hsing University, Taichung City (TW)

(72) Inventors: Chieh-Chen Huang, Taichung City (TW); Shou-Chen LO, Hsinchu City (TW); Dong-Yan Wu, Nantou County (TW); Jia-En WANG, Yilan City (TW); Shuo CHENG, Taoyuan City (TW); Guan-Min LI, Taichung City (TW); Yu-Han JIANG, Taichung City (TW); Tzu-Yu LIN, Taichung City (TW); Yu-Chieh CHEN, Taoyuan City (TW); Nai-Tzu KUO, Zhuqi Township (TW); Man-Yun YU, Taoyuan City (TW); Hsuan-Yu LIU, Taipei City (TW)

(21) Appl. No.: 15/986,559

(22) Filed: May 22, 2018

(30) Foreign Application Priority Data
May 23, 2017 (TW) 106116996

(51) Int. Cl. C12N 13/00; C12N 1/20; C12N 15/70; C12N 9/02; C12N 9/10

(52) U.S. Cl. CPC C12N 13/00; C12N 1/20; C12N 15/70; C12N 9/0008; C12Y 103/05001; C12N 9/1025; C12Y 203/03008; C12N 9/001; C12Y 102/07001

(57) ABSTRACT

The present invention provides a method for increasing the metabolic rate of recombinant microorganism growth under an anaerobic environment, wherein a recombinant strain is placed under an anaerobic environment and cultured under a culture condition, wherein the culture condition includes a potential difference and a nitrogen source, but not includes an organic carbon source. According to the method disclosed by the present invention, the recombinant strain can perform anaerobic respiration and metabolic reaction in an anaerobic environment, and can grow stably and rapidly.

### BACKGROUND OF THE INVENTION

### Field of the Invention

The present invention relates to a method for culturing a microorganism, and particularly relates to a method for increasing the growth and metabolic rate of growth of a recombinant microorganism in an anaerobic environment.

### Description of the Related Art

In recent years, the greenhouse effect has caused global climate and environmental problems to affect many countries seriously. The most important gas that causes the greenhouse effect is carbon dioxide. Therefore, in addition to efforts to promote reduction of carbon dioxide emissions in each country, it is more importantly to develop a method that can convert carbon dioxide into an available energy source. At present, many studies have found that the effect of carbon fixation can be achieved through E. coli or photosynthesis bacteria.

As E. coli has been previously disclosed by the literature as capable of using hydrogen, formic acid, lactic acid, etc. as electron providers, and using DMSO, puricic acid, nitrate, and nitrite as electron acceptors for anaerobic respiration. This means that E. coli can survive under anaerobic conditions by performing anaerobic respiration. Therefore, E. coli is more widely used as a carbon fixation platform than photosynthesis bacteria. However, although E. coli is a facultative anaerobic bacterium, it can survive under anaerobic conditions with anaerobic respiration, in fact, the growth of E. coli under anaerobic conditions is far inferior to the aerobic environment. Many biochemical reductase reactions need to work under anaerobic conditions, such as purpuric acid reduction reaction, hydrogenase reaction, nitrogen fixation reaction, carbon fixation reaction, etc.

Thus, it can be seen that Escherichia coli can not have good growth rate under anaerobic conditions. Since it cannot has a good metabolic rate in an anaerobic environment, a microbial platform based on Escherichia coli is difficult to be practically used at present.

### SUMMARY OF THE INVENTION

The main object of the present invention is to provide a method for increasing the growth and metabolic rate of recombinant microorganisms in an anaerobic environment, which enables the recombinant microorganisms to grow stably and rapidly in an environment that is anaerobic and has sufficient carbon dioxide.

Another object of the present invention is to provide a method for increasing the growth and metabolic rate of a recombinant microorganism in an anaerobic environment, which can make the recombinant microorganism have a good carbon fixation efficiency in an anaerobic and sugarless environment.

In order to achieve the above object, the disclosed an example of the present invention is a method for increasing the growth and metabolic rate of a recombinant microorganism in an anaerobic environment. A recombinant strain is placed under an anaerobic environment and cultured under a culture condition, wherein the culture condition includes a potential difference and a nitrogen source, and do not include organic carbon sources. According to the method disclosed by the present invention, the recombinant strain can perform anaerobic respiration and metabolic reaction in an anaerobic environment, and can grow stably and rapidly.

In one example of the invention, the genome of the recombinant strain includes an exogenous gene for expressing α-ketoglutarate:ferredoxin oxidoreductase.

Wherein the exogenous gene is korA lines and korB.

In another example of the present invention, the genome of the recombinant strain includes an exogenous gene and can express α-ketoglutarate: ferredoxin oxidoreductase, ATP-citrate lyase, fumarate reductase and succinate dehydrogenase.

Wherein the exogenous genes are korA, korB, aclA, aclB, frdA, frdB, frdC, sdhA, sdhB, and sdhC.

In an example of the present invention, the anaerobic environment refers to an environment having a carbon dioxide concentration of 0.2 to 50%.

In an example of the present invention, the potential difference is the difference in point potential between an electron provider and an electron acceptor in the anaerobic environment, wherein the electron provider is hydrogen, formic acid, lactic acid, glycerol, glycerol 3-phosphate NADH, or ATP, and the electron acceptor is dimethyl sulfoxide, trimethylamine oxide (TMAO), fumaric acid, nitrate, or nitrite.

In an example of the present invention, the electronic provider and the electronic receiver are each provided to the anaerobic environment at a predetermined concentration. For example, when the electron provider is hydrogen, the concentration thereof is 50 to 99%, and when the electron provider is formic acid, lactic acid, glycerin, glycerol 3-phosphate, NADH, or ATP, the concentration thereof is 0.2-20%; and the concentration of the electron acceptor is between 0.1 to 20%, and as an example, the concentration of dimethylarsine is 0.5% or more.

In an example of the present invention, the nitrogen source contains at least one amino acid. For example, the nitrogen source is a casein hydrolysate.

Further, another example of the present invention is a method for increasing the metabolic rate of growth of a recombinant carbon-fixing microorganism in an anaerobic environment by placing a recombinant strain capable of performing a reductive tricarboxylic acid cycle under an anaerobic environment and culturing with the above culture conditions, so that the recombinant bacteria can perform anaerobic respiration and metabolic reactions under anaerobic conditions, and can grow stably and rapidly.

### BRIEF DESCRIPTION OF THE DRAWINGS

FIG. 1 is a schematic diagram of a recombinant plastid map.

FIG. 2A is the result of the expression of the kor, ad, fr, and sdh genes by E. coli.

FIG. 2B is the result of the expression of KA, AF and FS fragments by E. coli.

FIG. 3 is the multiple of the 16S rRNA expression performance of each carbon fixation gene by E. coli.

FIG. 4 is the growth curve of anaerobic sugar-free culture of recombinant E. coli JM109.

FIG. 5 is a growth curve of recombinant Escherichia coli JM109 cultured in an anaerobic, sugar-free environment without dimethyl sulfoxide.

FIG. 6 is a growth curve of recombinant E. coli JM109 cultured in an anaerobic, sugar-free and carbon dioxide-free environment.

FIG. 7 is the growth curve of recombinant Escherichia coli JM109 cultured under the environment of anaerobic sugar-free and casein-free hydrolysate.

FIG. 8 is the growth curve of recombinant E. coli K12 cultured under anaerobic, sugar-free and carbon dioxide-free environment.

FIG. 9 is a growth curve of the recombinant Escherichia coli K12 cultured under the environment of anaerobic sugar-free and casein-free hydrolysate.

FIG. 10 is a growth curve of recombinant E. coli K12 cultured in an anaerobic, sugar-free environment wherein sodium nitrate is electron acceptor.

FIG. 11 shows the growth curves of recombinant KOR strains and blank recombinant strains cultured under anaerobic environment, respectively.

### DETAILED DESCRIPTION OF THE EMBODIMENTS

In the description of the present invention, scientific terms or phraseology, unless otherwise defined, are all construed in light of the understanding by those of ordinary skill in the art to which the present invention pertains.

The potential difference in the present invention refers to a difference between a point potential in an electron provider (reducing agent) and a point potential in an electron receiver (oxidant) in a culture environment. For example, the midpoint potential of hydrogen is −0.42 volts, the midpoint potential of dimethylarsine is +0.16 volts, the potential difference between them is 0.58 volts; the midpoint potential of hydrogen is −0.42 volts, and the midpoint potential of sodium nitrate is +0.42 volts and the potential difference between them is 0.84 volts.

The present invention discloses a method for increasing the metabolic rate of growth of a recombinant microorganism in an anaerobic environment by placing a recombinant microorganism in an anaerobic environment and incubating it under a predetermined culture condition. The condition contains a nitrogen source and a potential difference, and an organic carbon source such as glucose is not provided. By the method disclosed in the present invention, the recombinant microorganism can be stably grown in an anaerobic and sugar-free environment and undergo metabolic reaction.

Further, when the recombinant microorganism is subjected to anaerobic glucose-free culture by the method disclosed in the present invention, the recombinant microorganism can utilize the energy generated by the electron transfer in the culture environment, and the use of inorganic carbon sources is more efficient by performing carbon fixation by itself, that is, under the culture conditions disclosed in the present invention, enables the recombinant microorganism to have a chemically self-supporting capability in an environment without an organic carbon source, and has an excellent growth state.

Furthermore, the nitrogen source in the method disclosed in the present invention is an essential amino acid for providing growth of a recombinant microorganism. Specifically, the nitrogen source is preferably one containing amino acid such as glutamic acid/glutamine, proline and lysine, for example casein hydrolysate.

Hereinafter, some examples of the present invention will be described with reference to the accompanying drawings to illustrate the technical features and effects of the present invention.

### Example 1: Preparation of Recombinant E. coli (1)

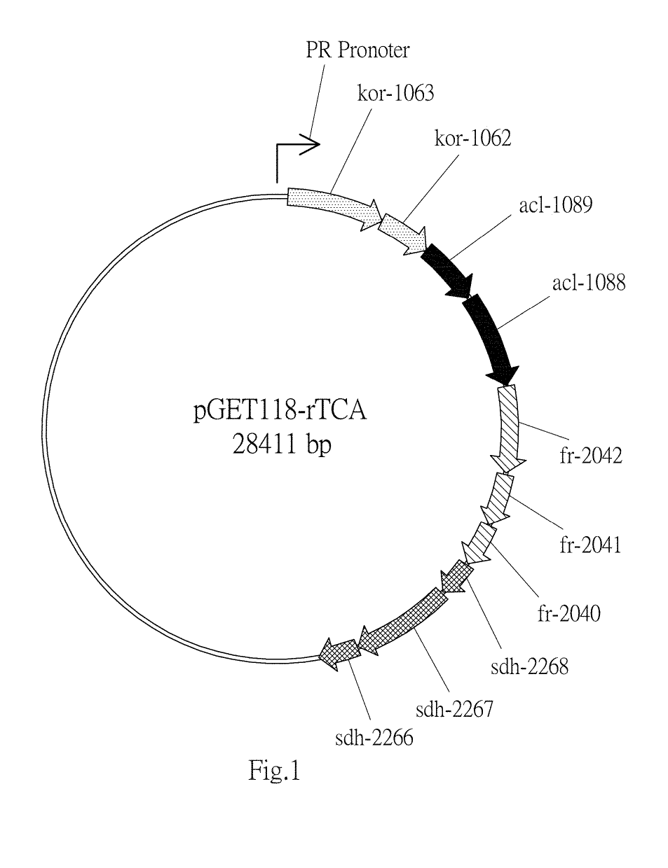

As shown in FIG. 1, Using OGAB method, kor, ad, fr and sdh fragments were constructed on the pGETS118 plastid to form a rTCA plasmid. The kor fragment which is 3028 bp in size and contains the genes korA and korB, is located after the PR promoter and is used to express α-ketoglutarate: ferredoxin oxidoreductase (hereinafter referred to as KOR); the acl fragment which is 3305 bp in size and contains the genes aclA and aclB, is located after fragment kor and is used to express ATP-citrate lyase (hereinafter referred to as ACL); the fr fragment has a size of 3627 bp and contains the genes frdA, frdB, and frdC and is located after the fragment ad to express fumarate reductase (hereinafter referred to as FR); The sdh fragment is 3559 bp in size and contains the amplified fragment of sdhA, sdhB and sdhC, and is used to express succinate dehydrogenase (SDH). The rTCA plasmid was further transferred to E. coli JM109 to obtain a recombinant E. coli JM109. Recombinant E. coli K12 was prepared in the same manner.

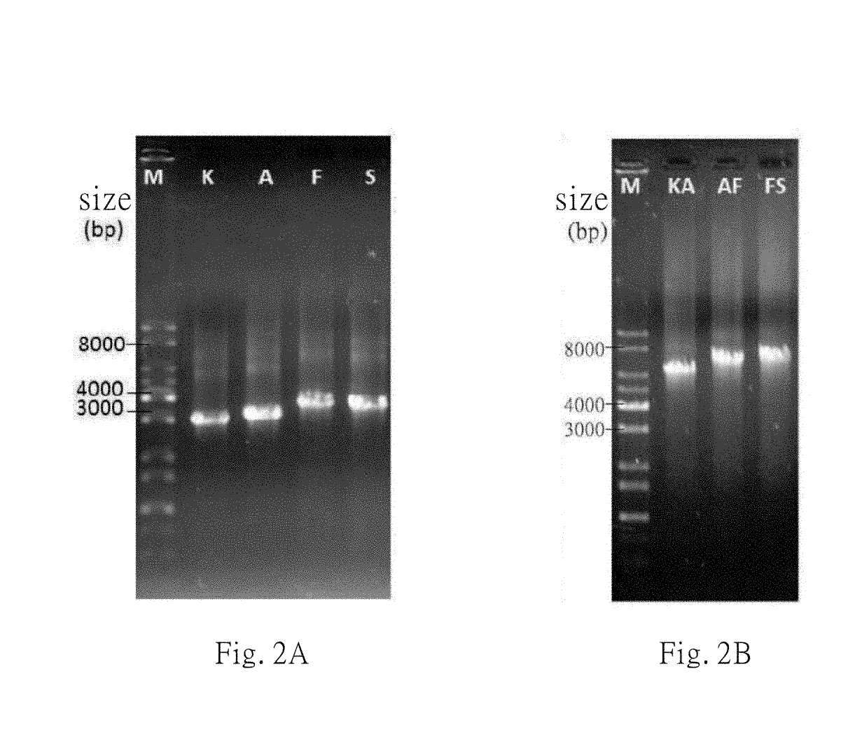

Further, the rTCA plastids in the recombinant E. coli JM109 were purified, and the kor, ad, fr, and sdh fragments were confirmed to be completely intact and in a predetermined sequence built on the rTCA plasmid, by specific primers corresponding to the kor, acl, fr, and sdh fragments, respectively, as shown in FIG. 2.

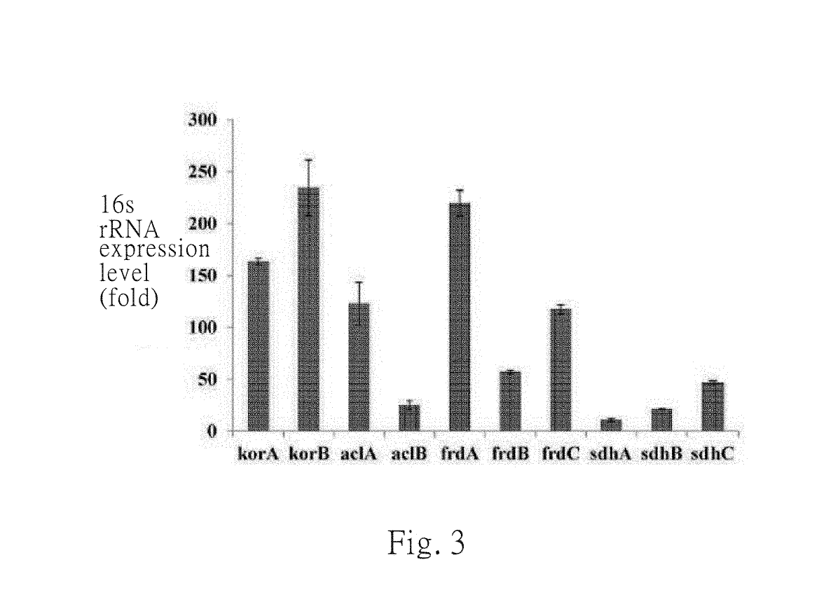

Furthermore, the expression of RNA in E. coli can be known by instant polymerase chain reaction. The results are shown in FIG. 3, showing that the expression amount of the transgenic genes in recombinant E. coli JM109 is about 10-245 times of that of 16S rRNA expression, that is, each of the genes is transcribed into RNA.

From the above, it is known that the transfer of carbon-fixation-related genes into E. coli can be achieved through the use of transgenic technology, making it a recombinant microorganism capable of expressing the enzymes required for the reductive tricarboxylic acid cycle and capable of carrying out the reductive tricarboxylic acid cycle within the microorganism.

### Example 2: Anaerobic Culture (1)

Recombinant Escherichia coli JM109 and Escherichia coli JM109 were cultured, respectively, wherein 0.2% glucose was added to M9 minimal medium in an aerobic environment before the strain entered the stationary phase, and then replaced into anaerobic culture.

The conditions for anaerobic cultivation are as follows: M9 minimal medium is used, organic carbon glucose is not added, and additional ions necessary for growth and metabolism of the strain are added, such as Mg2+, Ca2+, Fe2+, Ni3+, etc., 0.5% Dimethyl sulfoxide (DMSO) was used as an electron acceptor and 0.1% casein hydrolysate as a nitrogen source. The headspace was exposed to a mixture of hydrogen and carbon dioxide (9:1 v/v) as the electron source and inorganic carbon source.

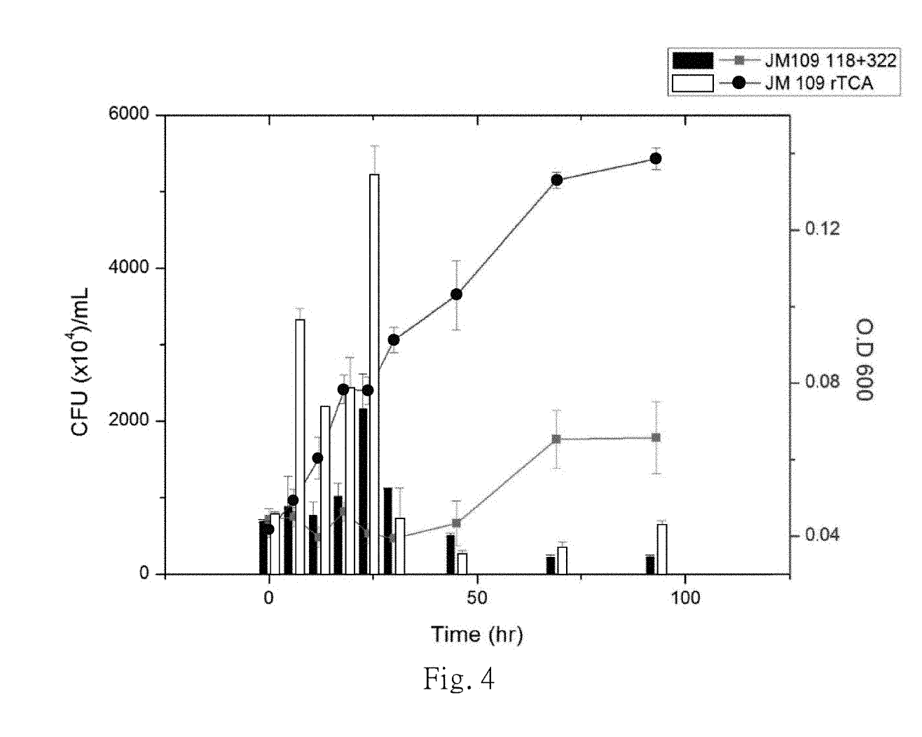

The culture results are shown in FIG. 4, showing that the recombinant Escherichia coli JM109 has a relatively fast growth under sugar-free culture, and the final OD value (OD value of about 0.15) is two times higher than that of the unreconstituted. E. coli (OD value about 0.07). Furthermore, it was more obvious from the colony count that the recombinant Escherichia coli JM109 reached the maximum number of colonies (about 5223×104 CFU/mL) in about 24 hours of culture and was significantly higher than the unreconstituted Escherichia coli JM109 (about 2160×10⁴ CFU/mL) (p value is 0.006634996), and the unreconstituted Escherichia coli JM109 was significantly reduced in the number of bacteria after about 45 hours of culture. In contrast, when the recombinant Escherichia coli JM109 was cultured for 93 hours, the number of colony was still about 652×10⁴ CFU/mL.

It can be seen from the above that the method disclosed by the present invention can effectively make the recombinant microorganism grow in a chemically self-supporting manner, and can utilize the inorganic carbon source more efficiently than the unrecombined microorganism, thereby significantly increasing the growth rate thereof.

### Example 3: Anaerobic Culture (2)

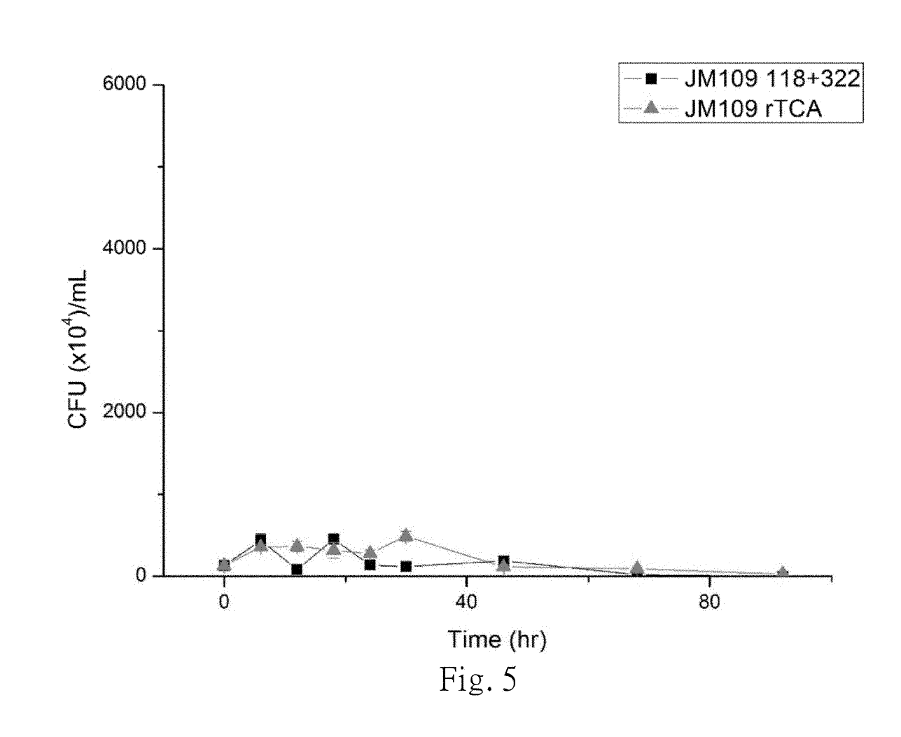

The culture method is basically the same as that described in Example 2, except that dimethyl sulfoxide is not added during anaerobic culture. The culture result is shown in FIG. 5.

From the results of FIG. 5, no growth tendency was observed in either the recombinant E. coli JM109 or the unrecombined E. coli JM109 under the culture conditions in which no dimethylsulfoxide was added. In other words, under anaerobic and sugar-free culture conditions, components that can act as electron acceptors should be added to provide energy for the reorganization of the carbon-fixing microorganisms to undergo carbon fixation, and for the growth of recombinant carbon-fixing microorganisms.

It can be seen from the above that the method disclosed in the present invention can indeed provide sufficient energy for the recombinant microorganism, so that the recombinant microorganism can still have good growth rate in an anaerobic and sugar-free environment.

### Example 4: Anaerobic Culture (3)

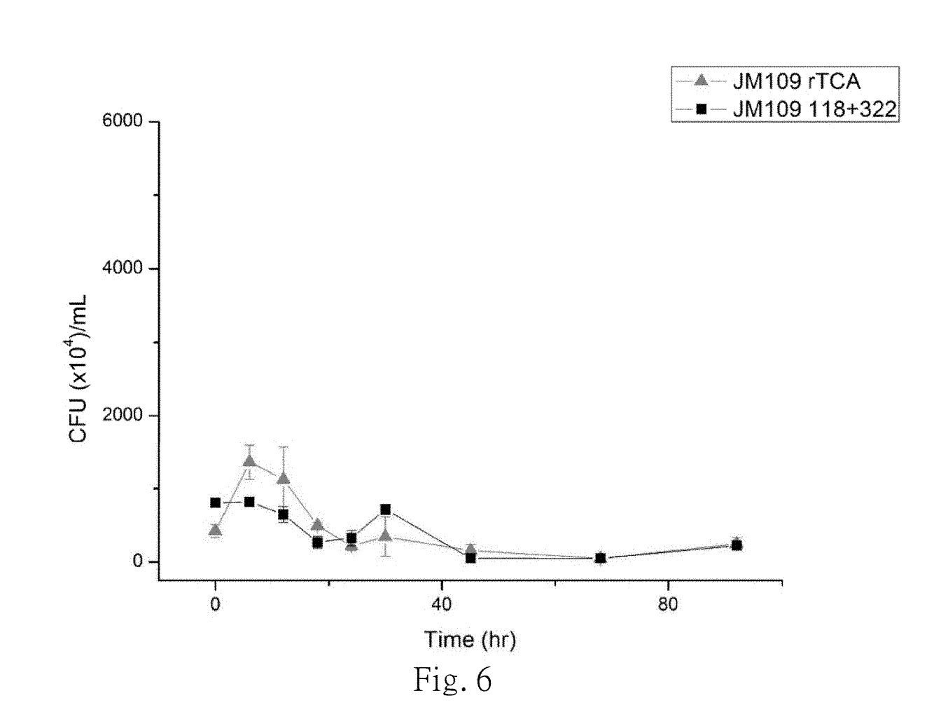

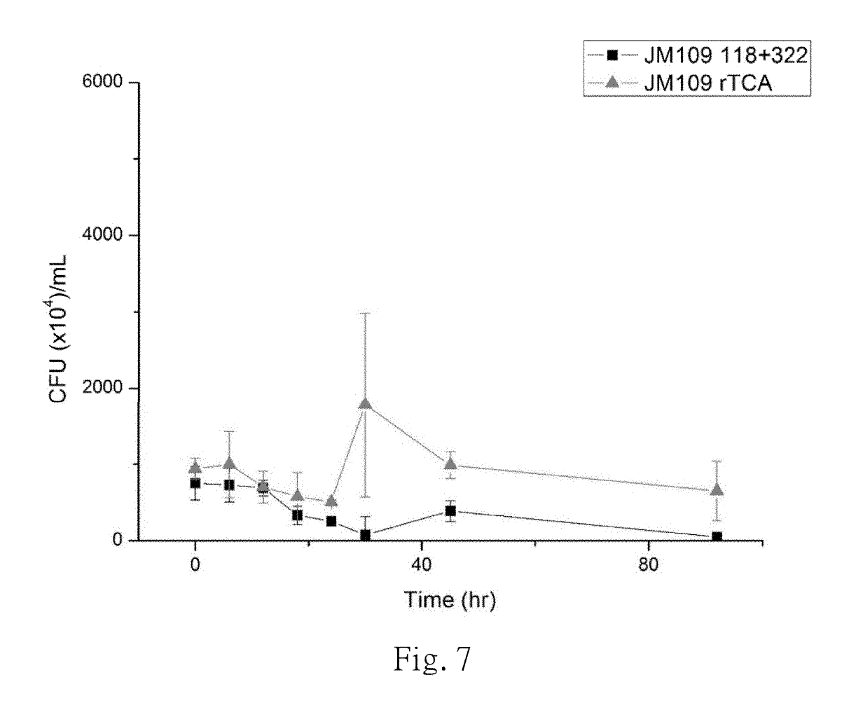

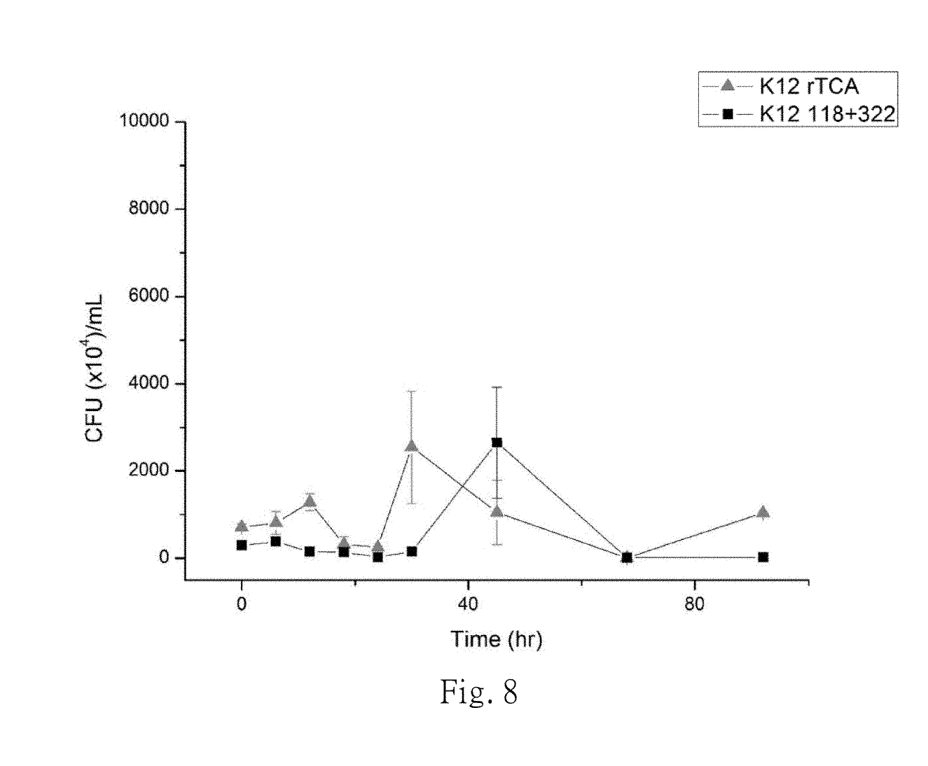

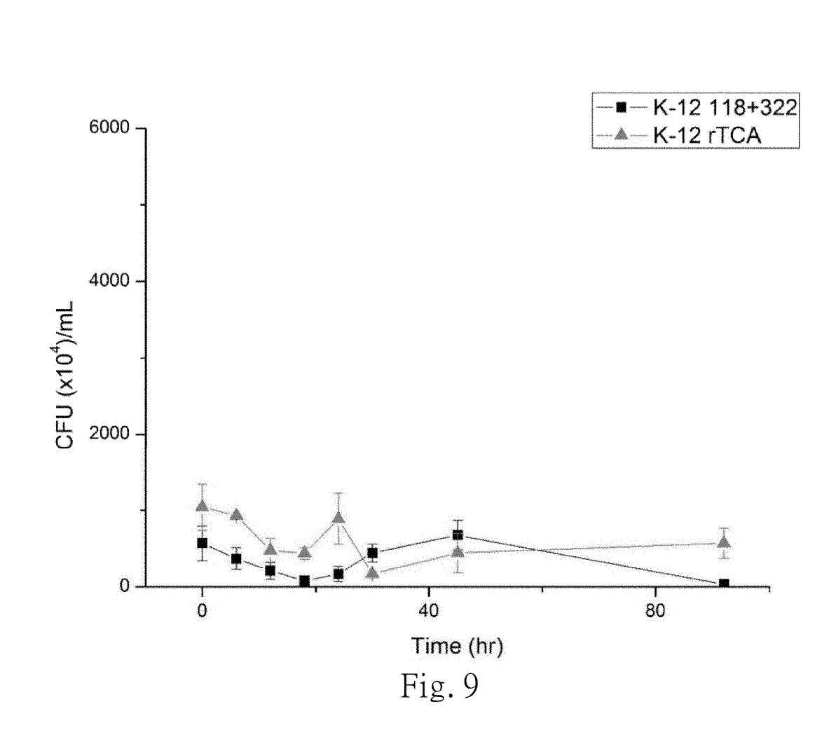

The culture method and conditions in this example is basically the same as that described in Example 2, except that in one of the culture groups, In the anaerobic culture, an additional 0.1% casein hydrolysate was added. The bottle headspace was exposed to hydrogen; the other culture group did not add casein hydrolysate and the headspace was exposed to a mixture of hydrogen and carbon dioxide (9:1 v/v). The recombinant E. coli JM109, the unrecombined E. coli JM109, the recombinant E. coli K12, and the unrecombined E. coli K12 were cultivated on the conditions of the respective culture groups, and the results are shown in FIGS. 6 to 9.

From the results of FIGS. 6 to 9, the growth curves of recombinant E. coli K12 and recombinant E. coli JM109 showed no tendency to grow under anaerobic, sugar-free and carbon dioxide-free culture conditions, indicating that the recombinant carbon-fixing microorganisms in an anaerobic, sugar-free culture environment needs carbon dioxide to be used as a carbon source. Further, under the anaerobic and sugar-free culture conditions, although carbon dioxide is provided as a carbon source, when there is no casein hydrolysate containing an amino acid, the recombinant carbon-fixing microorganism is still almost impossible to grow, indicating that in order for the recombinant carbon-fixing microorganism to grow well under anaerobic conditions, a source of amino acid must be provided.

Further, since casein hydrolysates contain high amounts of glutamic acid/glutamine, proline and lysine, From the results of this example, it can be deduced that the recombinant carbon-fixing microorganisms can have good growth rate in the environment of anaerobic culture, it is necessary to provide glutamic acid/glutamine, proline, glutamic acid, lysine or a combination of at least any two of the above amino acids.

### Example 5: Growth Ability Test

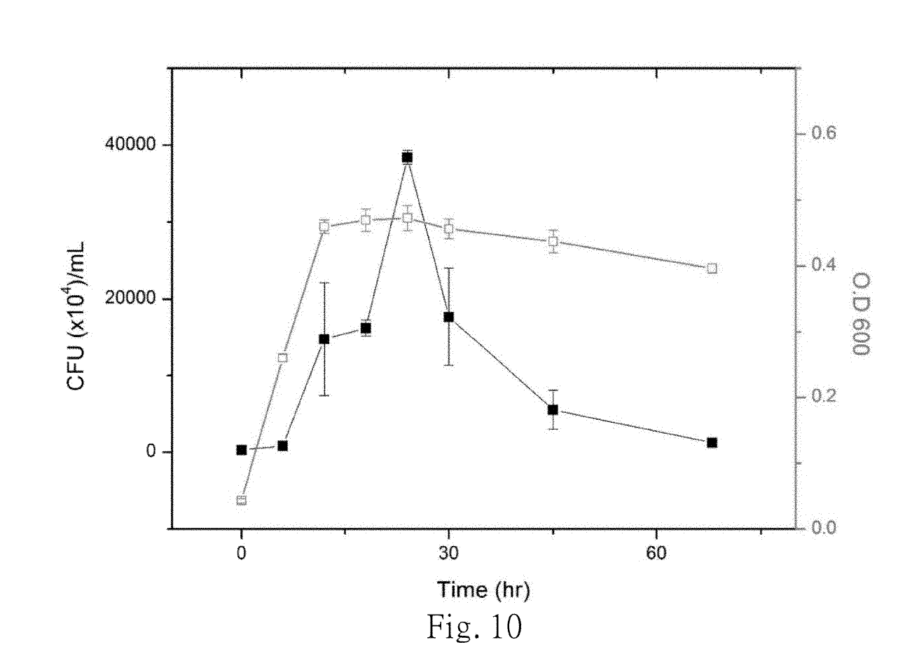

The culture method and conditions in this example is basically the same as that described in Example 2, except that the Dimethyl sulfoxide is replaced with sodium nitrate. The recombinant Escherichia coli K12 was subjected to anaerobic culture under these conditions, and the results are shown in FIG. 10.

As shown in FIG. 10, the recombinant E. coli K12 which was underwent anaerobic culture under the above-described culture conditions had a good growth state, and that after 24 hours of cultivation, the number of bacteria reached the highest growth amount (about 38400×10⁴ CFU/mL), OD rose rapidly in the first 12 hours, the highest amount reached 0.47. In comparison with recombinant E. coli K12, the amount of unrecombinant E. coli K12 measured at each time of the culture process was very low, and the number of colony was barely detectable after 24 hours of culture.

From this result, it can be seen that the energy source for the growth of recombinant microorganisms under anaerobic conditions is the energy generated when the electrons are transferred, and when the potential difference between the electron provider and the electron acceptor is greater, the energy that can be provided will increase more. So that the recombinant carbon-fixing microorganisms can obtain more energy for growth and have better growth efficiency.

### Example 6: Preparation of Recombinant Microorganisms (2)

Referring to the method disclosed in Example 1, korA and korB were constructed on pGETS118 plastids to form pGETS118 recombinant plastids so that pGETS118 recombinant plastids had kor fragments.

The pGETS118 recombinant plasmid and pGETS118 plasmid were respectively transferred into E. coli to form a recombinant KOR strain and a blank recombinant strain.

### Example 7: Anaerobic Culture (4)

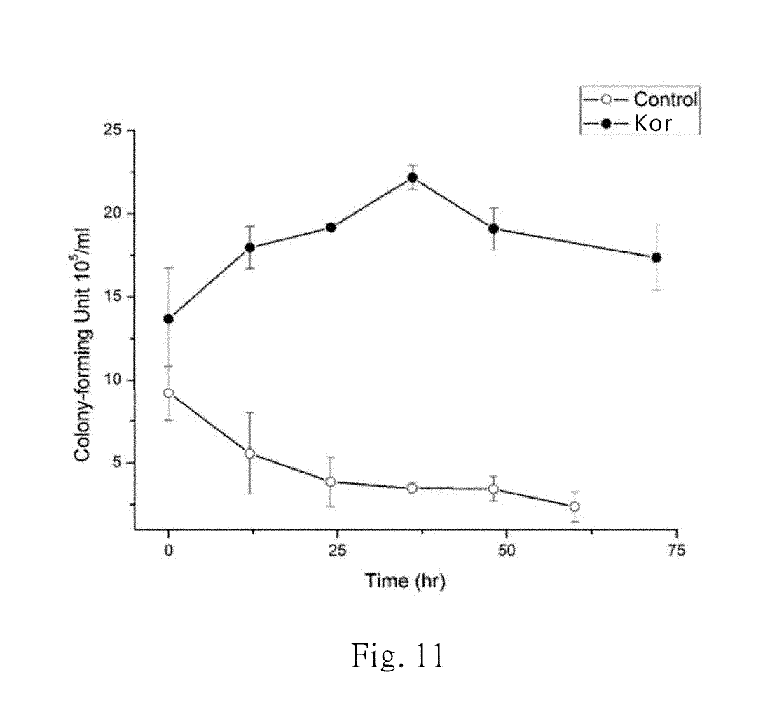

The recombinant KOR strain obtained in Example 6 and the blank recombinant strain were respectively cultured in an anaerobic medium. The composition of the anaerobic culture medium is shown in Table 1 below. Record the growth of each strain, as shown in FIG. 11.

TABLE 1
Composition of anaerobic medium Inoculation

| | total | 1000 | ml |
|---|---|---|---|
| Stocks | working conc. % (wt/vol) | add stock | |
| NaNO₃ | 0.01M | 10 | ml |
| Thiamine (Vit B1) | 0.00005% | 0.1 | ml |
| MgSO₄•7H2O | 0.002M | 2 | ml |
| NiCl₂ | 0.00001M | 1 | ml |
| FeCl₂•4H₂O | 0.00001M | 1 | ml |
| CaCl₂ | 0.0001M | 0.5 | ml |
| Chloramphenicol | 0.00002% | 1 | ml |
| 1X M9 | | 976.9 | ml |

| | |
|---|---|
| 90%H₂ + 10%CO₂ | Fill up the space |

From the results of FIG. 11, it can be seen that the recombinant KOR strain can maintain a high bacterial amount compared to the blank recombinant strain that has not been transferred into the kor gene fragment, showing that the method disclosed in the present invention does enable the growth of recombinant microorganisms in a chemically self-supporting manner and enhance the growth rate of recombinant microorganisms. Furthermore, even if the path of the reducing tricarboxylic acid cycle is not constructed, the recombinant microorganism can achieve the effect of growing efficiently under an anaerobic environment. As shown in this example here, only a single Kor gene fragment is necessary to be cloned into In E. coli, and the recombinant E. coli strains can grow in a chemically self-operated manner under anaerobic conditions.

The invention claimed is:

1. A method for increasing the metabolic rate of growth of a recombinant microorganism in an anaerobic environment, by placing a recombinant strain in an anaerobic environment and culturing under a culture condition wherein the culture condition includes a potential difference and a nitrogen source, but not includes organic carbon source.

2. A method for increasing the metabolic rate of growth of a recombinant microorganism in an anaerobic environment of claim 1, wherein the genome of the recombinant strain includes an exogenous gene and can express α-ketoglutamate acid: ferredoxin oxidoreductase.

3. A method for increasing the metabolic rate of growth of a recombinant microorganism in an anaerobic environment of claim 2, wherein the exogenous gene is korA and korB.

4. A method for increasing the metabolic rate of growth of a recombinant microorganism in an anaerobic environment of claim 1, wherein the genome of the recombinant strain includes an exogenous gene and can express can express α-ketoglutarate: ferredoxin oxidoreductase, ATP-citrate lyase, fumarate reductase and succinate dehydrogenase.

5. A method for increasing the metabolic rate of growth of a recombinant microorganism in an anaerobic environment of claim 4, wherein the exogenous genes are korA, korB, aclA, aclB, frdA, frdB, frdC, sdhA, sdhB, and sdhC.

6. A method for increasing the metabolic rate of growth of a recombinant microorganism in an anaerobic environment of claim 1, wherein the potential difference is a point potential difference between an electron provider and an electron acceptor in the anaerobic environment.

7. A method for increasing the metabolic rate of growth of a recombinant microorganism in an anaerobic environment of claim 6, the electron provider is selected from the group consisting of hydrogen, formic acid, lactic acid, glycerol, glycerol 3-phosphate, NADH, and ATP.

8. A method for increasing the metabolic rate of growth of a recombinant microorganism in an anaerobic environment of claim 7, wherein when the electron provider is hydrogen, the concentration is 50 to 99%.

9. A method for increasing the metabolic rate of growth of a recombinant microorganism in an anaerobic environment of claim 6, wherein the electron acceptor is selected from the group consisting of dimethyl sulfoxide, trimethylamine oxide, fumaric acid, nitrate, and nitrite.

10. A method for increasing the metabolic rate of growth of a recombinant microorganism in an anaerobic environment of claim 9, wherein the concentration of the electron acceptor is 0.1 to 20%.

11. A method for increasing the metabolic rate of growth of a recombinant microorganism in an anaerobic environment of claim 1, wherein the nitrogen source contains at least one amino acid.

12. A method for increasing the metabolic rate of growth of a recombinant microorganism in an anaerobic environment of claim 11, wherein the nitrogen source is a casein hydrolysate.

13. A method for increasing the metabolic rate of growth of a recombinant microorganism in an anaerobic environment of claim 1, wherein the anaerobic environment is an environment having a carbon dioxide concentration of 0.2 to 50%.
